(19) United States
(12) Patent Application Publication
(10) Pub. No.: US 2008/0194901 A1
(43) Pub. Date: Aug. 14, 2008

(54) Process To Prepare Two Iso Paraffinic Products From A Fischer-Tropsch Derived Feed

(76) Inventors: Michiel Cramwinckel, The Hague (NL); Jan Lodewijk Maria Dierickx, Amsterdam (NL); Arend Hoek, Amsterdam (NL)

Correspondence Address: SHELL OIL COMPANY, P O BOX 2463, HOUSTON, TX, 772522463, US

(21) Appl. No.: 11/793,792

(22) PCT Filed: Dec. 21, 2005
(86) PCT No.: PCT/EP2005/057012

§ 371 Date: Nov. 27, 2007

(30) Foreign Application Priority Data
Dec. 23, 2004 (EP) 04106941.0

(51) Int. Cl. C07C 5/00

(52) U.S. Cl. 585/734

(57) ABSTRACT

Process to prepare an iso-paraffinic product having a carbon range of Cx to Cy and a iso-paraffinic product having a carbon range of Cn to Cm from a Fischer-Tropsch derived feed by performing the following steps, (a) obtaining from the Fischer-Tropsch derived feed at least two different compositions (i) and (ii), which composition (i) has a greater fraction of compounds in the carbon range of C2n to C2m than composition (ii) and composition (ii) has a greater content of C2x to C2y than composition (i); (b) performing separately a hydroconversion/hydroisomerisation step on feed compositions (i) and (ii) and isolating from the thus obtained effluents the iso-paraffinic product having a carbon range of Cx to Cy and the iso-paraffinic product having a carbon range of Cn to Cm.

### FIELD OF THE INVENTION

The invention is directed to a process to prepare at least two different iso-paraffinic products from a Fischer-Tropsch derived synthesis product.

### BACKGROUND OF THE INVENTION

WO-A-02070629 describes a process to prepare at least two iso-paraffinic products from a Fischer-Tropsch derived wax. This publication describes a process to prepare a gas oil product and a base oil product from a Fischer-Tropsch derived synthesis product by performing a hydroconversion/hydroisomerisation step on a heavy wax and isolation of a gas oil fraction and a residue from the obtained cracked effluent. The gas oil as obtained had an iso-paraffin content of 80 wt %. The residue is further distilled to obtain a distillate fraction boiling between 370 and 510° C. This fraction boiling between 370 and 510° C. was subjected to a catalytic dewaxing step to obtain various iso-paraffinic base oil grades.

It is observed that when waxy normal-paraffinic feeds, as for example the Fischer-Tropsch waxes, are subjected to a hydroconversion/hydroisomerisation step it is possible to optimise the quality, i.e. the content of iso-paraffins, and yield for one boiling fraction only.

The object of the present invention is to optimise the hydroconversion and hydroisomerisation step of Fischer-Tropsch derived waxy feed in such a manner that the yield and quality of two or more boiling fractions, i.e. products, can be optimised.

### SUMMARY OF THE INVENTION

The following process solves the above problem. Process to prepare an iso-paraffinic product having a carbon range of Cx to Cy and an iso-paraffinic product having a carbon range of Cn to Cm from a Fischer-Tropsch derived feed by performing the following steps,

(a) obtaining from the Fischer-Tropsch derived feed at least two different compositions (i) and (ii), which composition (i) has a greater fraction of compounds in the carbon range of C2n to C2m than composition (ii) and composition (ii) has a greater content of C2x to C2y than composition (i), each fraction containing at least 5 wt % based on the whole fraction of material boiling above 370° C.;
(b) performing separately a hydroconversion/hydroisomerisation step on feed compositions (i) and (ii) and isolating from the thus obtained effluents the iso-paraffinic product having a carbon range of Cx to Cy and the iso-paraffinic product having a carbon range of Cn to Cm.

Applicants found that by performing the hydroconversion/hydroisomerisation step on a feed which is rich in a fraction having substantially the double number of carbon atoms than the desired iso-paraffin product a more optimal process in terms of yield and iso-paraffin content is achieved. By performing the hydroconversion/hydroisomerisation step separately on the different feeds it is possible to optimise yield and quality for every iso-paraffinic product made from the Fischer-Tropsch waxy product.

### DETAILED DESCRIPTION OF THE INVENTION

The Fischer-Tropsch derived feed can be obtained by well-known processes, for example the so-called commercial Slurry Phase Distillate technology of Sasol, the Shell Middle Distillate Synthesis Process or by the “AGC-21” Exxon Mobil process. These and other processes are for example described in more detail in EP-A-776959, EP-A-668342, U.S. Pat. No. 4,943,672, U.S. Pat. No. 5,059,299, WO-A-9934917 and WO-A-9920720. Typically these Fischer-Tropsch synthesis products will comprise hydrocarbons having 1 to 100 and even more than 100 carbon atoms. This hydrocarbon product will comprise normal paraffins, iso-paraffins, oxygenated products and unsaturated products. If base oils are one of the desired iso-paraffinic products it may be advantageous to use a relatively heavy Fischer-Tropsch derived feed. The relatively heavy Fischer-Tropsch derived feed has at least 30 wt %, preferably at least 50 wt %, and more preferably at least 55 wt % of compounds having at least 30 carbon atoms. Furthermore the weight ratio of compounds having at least 60 or more carbon atoms and compounds having at least 30 carbon atoms of the Fischer-Tropsch derived feed is preferably at least 0.2, more preferably at least 0.4 and most preferably at least 0.55. Preferably the Fischer-Tropsch derived feed comprises a C₂₀⁺ fraction having an ASF-alpha value (Anderson-Schulz-Flory chain growth factor) of at least 0.925, preferably at least 0.935, more preferably at least 0.945, even more preferably at least 0.955. The ASF alpha value is suitably derived from the fractions containing the C₂₀-compounds and the C₄₀-compounds. A very suitable method comprises hydrogenation and gas chromatography. Such a Fischer-Tropsch derived feed can be obtained by any process, which yields a relatively heavy Fischer-Tropsch product as described above. Not all Fischer-Tropsch processes yield such a heavy product. An example of a suitable Fischer-Tropsch process is described in WO-A-9934917.

The Fischer-Tropsch derived feed will contain no or very little sulphur and nitrogen containing compounds. This is typical for a product derived from a Fischer-Tropsch reaction, which uses synthesis gas containing almost no impurities. Sulphur and nitrogen levels will generally be below the detection limits, which are currently 5 ppm for sulphur and 1 ppm for nitrogen respectively.

The process of the present invention is directed to prepare two iso-paraffinic products, one having a carbon range of Cx to Cy and one having a carbon range of Cn to Cm. In these carbon ranges x<y and n<m, while x>n. Suitably the difference between x and y is between 10 and 35, more suitably between 15 and 30 for the values of 15<x<30 and 30<y<55. The difference between x and y is suitably between 0 and 15, more suitably between 4 and 11 for the values of 12<x<20 and 18<y<27. The difference between n and m is suitably between 2 and 15, more suitably between 4 and 11, for the values 12<n<18 and 18<m<28. The difference for n and m is suitably between 2 and 12, more suitably between 4 and 11 for the values 5<n<14 and 7<m<20. With having a carbon range is here meant that more than 80 wt % of the product comprises of compounds having a number of carbon atoms in said range. More preferably more than 95 wt % of the iso-paraffinic product comprises of compounds having a number of carbon atoms in said range.

In a preferred embodiment n is between 14 and 16, m is between 20 and 25, x is between 20 and 25 and y is between 40 and 50. The resultant iso-paraffinic products will boil in the respective gas oil range and base oil range. The iso-paraffin content of the gas oil product may be expressed in its pour point wherein the lower the pour point the higher the iso-paraffin content. The iso-paraffin content of the product boiling in the base oil range can be expressed in its wax content as measured by solvent dewaxing at −20° C. The lower the wax content the higher the iso-paraffin content. It is of course understood that any residual wax in the iso-paraffinic product is suitably removed by optional solvent or catalytic dewaxing. Dewaxing can thus further optimize the iso-paraffin content of the higher boiling iso-paraffinic products obtainable by the process according to the present invention.

In another embodiment of the present invention n is between 10 and 12, m is between 14 and 16, x is between 14 and 16 and y is between 20 and 25. The resultant iso-paraffinic products will boil in the respective kerosene range and gas oil range. The iso-paraffin content of the product boiling in the kerosene range can be expressed in its freeze point, wherein a low freeze point is indicative for a high iso-paraffinic content.

In another embodiment of the present invention n is 5, m is between 9 and 12, x is between 14 and 16 and y is between 20 and 25. The resultant iso-paraffinic products will boil in the respective naphtha range and gas oil range. The iso-paraffin content of the naphtha type of product may be analyzed by means of gas chromatography.

In a next embodiment of the present invention, n is between 9 and 12 m is between 16 and 20, x is between 16 and 20 and y is between 20 and 25. The two resultant iso-paraffin products both boil in the gas oil range and may be advantageously be combined resulting in a gas oil product which has an optimal content of iso-paraffins in both its high boiling as well as its low boiling part. The high content of the iso-paraffins in the high boiling part is advantageous because a gas oil may then be prepared having both a higher density, a higher T95 wt % boiling point combined with improved cold flow properties, e.g. a low cloud point.

Step (a) may be performed in any manner which results in that from the Fischer-Tropsch derived feed at least two different compositions (i) and (ii) are obtained, which composition (ii) has a greater fraction of compounds in the carbon range of C2x to C2y than composition (i) and composition (i) has a greater content of C2n to C2m than composition (ii). As an example, with Cx and C2x is here meant x carbons and 2 times x carbons. More preferably the weight ratio of C2n to C2m over Cn to Cm is greater than 1.5, even more preferably greater than 2 in composition (i). More preferably the weight ratio of C2x to C2y over Cx to Cy is greater than 1.5, even more preferably greater than 2 in composition (ii).

Suitably the at least two different compositions (i) and (ii) contain a fraction boiling above 370° C. especially above 540° C. For the lighter fraction(s) the amount is at least 5 wt % of the total fraction, suitably at least 10 wt %, preferably 12-80 wt %, more preferably 15-65 wt % of material boiling above 370° C. The lighter fraction suitably contains at least 3 wt %, more suitably at least 6 wt %, preferably 10-65 wt %, more preferably 15-55 wt % of material boiling above 540° C. A heavier product results is increased isomerisation. For the heavier product the amount is suitably at least 10 wt % of the total fraction, more suitably at least 15 wt %, preferably 20-100 wt % of material boiling above 370° C. The heavier fraction suitably contains at least 5 wt %, more suitably at least 10 wt %, preferably 15-95 wt %, more preferably 30-90 wt % of material boiling above 540° C.

Below some preferred embodiments for step (a) will be described which alone or in combination with one of the other embodiments result in a preferred manner of performing step (a).

Fischer-Tropsch synthesis is suitably performed in two or more parallel-operated reactors in the presence of a suitable catalyst on a feed comprising of hydrogen and carbon monoxide. These Fischer-Tropsch reactors are well known and may be so-called fixed bed reactors or slurry type reactors. The paraffinic product as obtained in such a reactor is typically obtained as a separate gaseous fraction and a liquid wax fraction. The gaseous products are typically condensed and combined with the liquid wax product. In the present case the condensed products are preferably added in a greater amount to composition (i) than to composition (ii). This will result in that relative composition (i) will comprise more low boiling compounds than composition (ii).

Another possible method of performing step (a) is by performing some of the parallel operated Fischer-Tropsch reactors differently than the other reactors thereby obtaining a Fischer-Tropsch product comprising a C₂₀⁺ fraction having different ASF-alpha values (Anderson-Schulz-Flory chain growth factor). This can be achieved by variation of for example pressure, temperature and/or residence time or by using different catalyst types. By providing composition (i) with more of the Fischer-Tropsch product having the lower ASF-alpha value and composition (ii) with the Fischer-Tropsch product having the higher ASF-alpha value the desired difference as described in the claims is achieved. Preferably the ASF-alpha value of the Fischer-Tropsch product provided to composition (i) is below 0.94, e.g. between 0.90 and 0.93 and the ASF-alpha value of the Fischer-Tropsch product provided to composition (ii) is greater than 0.94, e.g. between 0.95 and 0.98.

In another embodiment for step (a) the Fischer-Tropsch product may be separated into a high and low boiling fraction by means of suitably distillation or flashing. A disadvantage of such a method is that it requires substantial amount of energy to separate this feed. In a preferred embodiment for step (a) the Fischer-Tropsch product may be split into three parts (aa,bb,cc), wherein one part (aa) is separated into a high boiling part and a lower boiling part by means of suitably distillation or flashing. By adding the lower boiling part to part (bb) and the high boiling part to (cc) compositions (i) and (ii) are obtained respectively. This embodiment requires less energy than splitting the entire feed while at the same time the advantages of the present invention are still achieved.

In another preferred embodiment of the present invention use is made of the so-called slops that are obtained as off-spec products in a typical gas-to-liquids process. Sources of such slops may be for example off-spec wax products or off-spec products of the hydroconversion/hydroisomerisation step. Such off spec products are made for example at start up conditions, process failures, distillation column upsets and other unusual conditions. Slops are preferably collected in slob tanks. Preferably liquid and solid slops are collected separately. Liquid slops, also referred to as cold slops, are liquid at room temperature and solid slops, also referred to as hot slops, have to be heated to keep them liquid at ambient conditions. By adding the cold slops to the Fischer-Tropsch feed a composition (i) is suitably obtained and/or by adding the hot slops to another part of the Fischer-Tropsch feed a composition (ii) is suitably obtained.

In another embodiment of the present invention step (a) is performed by selectively adding part or all of the unconverted fraction obtained in step (b) to the Fischer-Tropsch feed to obtain composition (ii). Preferably the unconverted products of the hydroconversion/hydroisomerisation of composition (ii) are used to prepare composition (ii). More preferably the unconverted products of feed composition (i) are used to make composition (ii). This will further increase the fraction of compounds having double the amount of carbon atoms in composition (ii) making the feed excellent for use in preparing the relatively more heavy iso-paraffinic products, for example the base oils and the heavy gas oils.

Prior to the hydroconversion/hydroisomerisation step (b) the feed compositions may optionally be subjected to a mild hydrotreatment step, in order to remove any oxygenates and saturate any olefinic compounds present in the reaction product of the Fischer-Tropsch reaction. Preferably the hydrogenation step reduces the level of oxygenates to below 150 ppm as measured by infrared absorption spectrometry and reduces the level of unsaturated compounds to below the detection limit of the infrared absorption spectrometry.

Such a hydrotreatment is for example described in EP-B-668342. The mildness of the hydrotreating step is preferably expressed in that the degree of conversion in this step is less than 20 wt % and more preferably less than 10 wt %. The conversion is here defined as the weight percentage of the feed boiling above 370° C., which reacts to a fraction boiling below 370° C. After such a mild hydrotreatment, lower boiling compounds, having four or less carbon atoms and other compounds boiling in that range, will preferably be removed from the effluent before it is used in step (b). Examples of suitable catalysts are noble metal catalyst as for example platinum based hydrogenation catalysts or non-noble catalysts such as high content nickel catalysts.

The initial boiling point of the compositions (i) and (ii) may suitably range from the boiling point of pentane and up to 500° C. The initial boiling points of both compositions may be the same or different. If these IBP values are different then it is preferred that the initial boiling point of composition (i) is lower than the initial boiling point of composition (ii).

The feed compositions (i) and (ii) for step (b) may next to the Fischer-Tropsch derived feed also comprise of mineral crude derived fractions and/or gas field condensates. These additional sulphur containing co-feeds are advantageous when a sulphided catalyst is used in step (b). The sulphur in the feed will keep the catalyst in its sulphided form. The sulphur may be removed in a down stream treating unit or, in case the quantities are very low, become part of the product of the present invention.

The hydroconversion/hydroisomerisation reaction of step (b) is preferably performed in the presence of hydrogen and a catalyst, which catalyst can be chosen from those known to one skilled in the art as being suitable for this reaction of which some will be described in more detail below. The catalyst may in principle be any catalyst known in the art to be suitable for isomerising paraffinic molecules. In general, suitable hydroconversion/hydroisomerisation catalysts are those comprising a hydrogenation component supported on a refractory oxide carrier, such as amorphous silica-alumina (ASA), alumina, fluorided alumina, molecular sieves (zeolites) or mixtures of two or more of these. One type of preferred catalysts to be applied in the hydroconversion/hydroisomerisation step in accordance with the present invention are hydroconversion/hydroisomerisation catalysts comprising platinum and/or palladium as the hydrogenation component. A very much preferred hydroconversion/hydroisomerisation catalyst comprises platinum and palladium supported on an amorphous silica-alumina (ASA) carrier. The platinum and/or palladium is suitably present in an amount of from 0.1 to 5.0% by weight, more suitably from 0.2 to 2.0% by weight, calculated as element and based on total weight of carrier. If both present, the weight ratio of platinum to palladium may vary within wide limits, but suitably is in the range of from 0.05 to 10, more suitably 0.1 to 5. Examples of suitable noble metal on ASA catalysts are, for instance, disclosed in WO-A-9410264 and EP-A-0582347. Other suitable noble metal-based catalysts, such as platinum on a fluorided alumina carrier, are disclosed in e.g. U.S. Pat. No. 5,059,299 and WO-A-9220759.

A second type of suitable hydroconversion/hydroisomerisation catalysts are those comprising at least one Group VIB metal, preferably tungsten and/or molybdenum, and at least one non-noble Group VIII metal, preferably nickel and/or cobalt, as the hydrogenation component. Both metals may be present as oxides, sulphides or a combination thereof. The Group VIB metal is suitably present in an amount of from 1 to 35% by weight, more suitably from 5 to 30% by weight, calculated as element and based on total weight of the carrier. The non-noble Group VIII metal is suitably present in an amount of from 1 to 25 wt %, preferably 2 to 15 wt %, calculated as element and based on total weight of carrier. A hydroconversion catalyst of this type which has been found particularly suitable is a catalyst comprising nickel and tungsten supported on fluorided alumina.

The above non-noble metal-based catalysts are preferably used in their sulphided form. In order to maintain the sulphided form of the catalyst during use some sulphur needs to be present in the feed. Preferably at least 10 ppm and more preferably between 50 and 150 ppm of sulphur is present in the feed.

A preferred catalyst, which can be used in a non-sulphided form, comprises a non-noble Group VIII metal, e.g., iron, nickel, in conjunction with a Group IB metal, e.g., copper, supported on an acidic support. Copper is preferably present to suppress hydrogenolysis of paraffins to methane. The catalyst has a pore volume preferably in the range of 0.35 to 1.10 ml/g as determined by water absorption, a surface area of preferably between 200-500 m²/g as determined by BET nitrogen adsorption, and a bulk density of between 0.4-1.0 g/ml. The catalyst support is preferably made of an amorphous silica-alumina wherein the alumina may be present within wide range of between 5 and 96 wt %, preferably between 20 and 85 wt %. The silica content as SiO₂ is preferably between 15 and 80 wt %. Also, the support may contain small amounts, e.g., 20-30 wt %, of a binder, e.g., alumina, silica, Group IVA metal oxides, and various types of clays, magnesia, etc., preferably alumina or silica.

The preparation of amorphous silica-alumina microspheres has been described in Ryland, Lloyd B., Tamele, M. W., and Wilson, J. N., Cracking Catalysts, Catalysis: volume VII, Ed. Paul H. Emmett, Reinhold Publishing Corporation, New York, 1960, pp. 5-9.

The catalyst is prepared by co-impregnating the metals from solutions onto the support, drying at 100-150° C., and calcining in air at 200-550° C. The Group VIII metal is present in amounts of about 15 wt % or less, preferably 1-12 wt %, while the Group IB metal is usually present in lesser amounts, e.g., 1:2 to about 1:20 weight ratio respecting the Group VIII metal.

A typical catalyst is shown below:

| Ni, wt % | 2.5-3.5 | |
|---|---|---|
| Cu, wt % | 0.25-0.35 | |
| Al₂O₃—SiO2 wt % | 65-75 | |
| Al₂O₃ (binder) wt % | 25-30 | |
| Surface Area | 290-325 | m²/g |
| Pore Volume (Hg) | 0.35-0.45 | ml/g |
| Bulk Density | 0.58-0.68 | g/ml |

Another class of suitable hydroconversion/hydroisomerisation catalysts are those based on zeolitic materials, suitably comprising at least one Group VIII metal component, preferably Pt and/or Pd, as the hydrogenation component. Suitable zeolitic and other aluminosilicate materials, then, include Zeolite beta, Zeolite Y, Ultra Stable Y, ZSM-5, ZSM-12, ZSM-22, ZSM-23, ZSM-48, MCM-68, ZSM-35, SSZ-32, ferrierite, mordenite and silica-aluminophosphates, such as SAPO-11 and SAPO-31. Examples of suitable hydroisomerisation/hydroisomerisation catalysts are, for instance, described in WO-A-9201657.

The above catalysts are preferably reduced before being used. The metallic catalyst may be obtained as an oxidic or a pre-reduced catalyst. The above catalysts which are used in a sulphided form may be obtained in a oxidic, a pre-sulphided or a presulphurised form. Preferably the start-up procedure of the catalyst manufacturer is followed. Pre-reducing the catalyst for use in a metallic form may also be achieved in situ by reducing the catalyst by contacting with hydrogen. Preferably the contacting is achieved by contacting the catalyst at an elevated temperature with a hydrogen in e.g. nitrogen mixture stream. More preferably the hydrogen content is increased over time and/or the temperature is gradually increased. A skilled person will be able to achieve a successful reduction of the catalyst by applying generally applied skills.

In step (b) the feed is contacted with hydrogen in the presence of the catalyst at elevated temperature and pressure. The temperatures typically will be in the range of from 175 to 425° C., preferably higher than 250° C. and more preferably from 280 to 400° C. The hydrogen partial pressure will typically be in the range of from 10 to 250 bar and preferably between 20 and 100 bar. The hydrocarbon feed may be provided at a weight hourly space velocity of from 0.1 to 5 kg/l/hr (mass feed/volume catalyst bed/time), preferably higher than 0.5 kg/l/hr and more preferably lower than 2 kg/l/hr. Hydrogen may be supplied at a ratio of hydrogen to hydrocarbon feed from 100 to 5000 Nl/kg and preferably from 250 to 2500 Nl/kg.

Step (b) is preferably performed in a reactor provided with beds of the heterogeneous catalyst as described above. More preferably step (b) is performed in two parallel and continuously operated reactors for respectively feed composition (i) and (ii). Preferably the two reactors have the same size. Preferably the reactors have the same type of catalyst. It is of course understood that embodiments with more than two parallel operated reactors are also embodiments of the present invention, provided that the feed composition to at least two of said reactors are different according to the process of the present invention as described above.

The conversion in (b), which is defined as the weight percentage of the feed boiling above 370° C. which reacts per pass to a fraction boiling below 370° C., is at least 20 wt %, preferably at least 25 wt %, but preferably not more than 90 wt %. The conversion may be the same for feed composition (i) and (ii) or different. In a preferred embodiment the conversion for the different feed compositions is optimised in order to achieve the desired yield and quality for each different iso-paraffinic product. Preferably the conversion as defined above is higher when performing step (b) for composition (i) than when performing step (b) for composition (ii). The difference in conversion between feed compositions (i) and (ii) is preferably more than 5 wt %, more preferably more than 10 wt % and even more preferably more than 15 wt %. The difference will at most be preferably 30 wt %. Preferably the conversion in step (b) for composition (ii) is between 30 and 60 wt % and the conversion in step (b) for composition (i) is between 50 and 90 wt %. The feed as used above in the definition is the total hydrocarbon feed fed to step (b), thus also any optional recycle of the unconverted products of respectively feed compositions (i) and (ii).

From the effluents of step (b) as obtained from feed compositions (i) and (ii) the iso-paraffinic products may suitably be isolated by means of distillate fractionation. This may be performed on the combined effluent or separately. If the distillation is performed separately it is possible to obtain for the same boiling fraction a highly iso-paraffinic product and a less highly iso-paraffinic product. This may be advantageous in cases that for both products separate applications are foreseen. For example a gas oil having a low pour point may find application as a diesel blending component or as a drilling fluid component while the gas oil having a higher pour point and lower iso-paraffin content may find application as a steam cracker feedstock to prepare selectively ethylene. Also highly isomerised naphtha products may find application as gasoline-blending component while the less isomerised naphtha's can find applications as solvents or also as steam cracker feedstocks. The distillation can be performed at atmospheric pressure to isolate the middle distillate fractions and a residue boiling in the base oil range. The residue may optionally be further distilled under vacuum conditions in order to remove the very high boiling fraction, i.e. the unconverted compounds as described above, which may find application as compounds to increase the content of compounds having C2x to C2y carbons in composition (ii).

The iso-paraffinic product boiling in the base oil range is preferably further dewaxed in order to remove any residual normal paraffins. The pour point reducing step may be a solvent dewaxing treatment. Preferably this treatment is a catalytic pour point reducing treatment step. With the catalytic pour point reducing treatment is understood every process wherein the pour point, as measured by ASTM D 97, of the base oil is reduced by more than 10° C., preferably more than 20° C., more preferably more than 25° C.

The catalytic pour point reducing process can be performed by any process wherein, in the presence of a catalyst and hydrogen the pour point of the fraction after processing is improved, as specified above. Suitable dewaxing catalysts are heterogeneous catalysts comprising a molecular sieve optionally in combination with a metal having a hydrogenation function, such as the Group VIII metals. Preferred molecular sieves are intermediate pore size zeolites. Preferably the intermediate pore size zeolites have a pore diameter of between 0.35 and 0.8 nm. Suitable intermediate pore size zeolites and other aluminosilicate materials are zeolite beta mordenite, ZSM-5, ZSM-12, ZSM-22, ZSM-23, MCM-68, SSZ-32, ZSM-35 and ZSM-48. Another preferred group of molecular sieves are the silica-aluminophosphate (SAPO) materials of which SAPO-11 is most preferred as for example described in U.S. Pat. No. 4,859,311. ZSM-5 may optionally be used in its HZSM-5 form in the absence of any Group VIII metal. The other molecular sieves are preferably used in combination with an added Group VIII metal, or mixtures of said metals. Suitable Group VIII metals are nickel, cobalt, platinum and palladium. Examples of possible combinations are Pt/Zeolite beta, PtPd/Zeolite beta, Ni/ZSM-5, Pt/ZSM-23, Pd/ZSM-23, Pt/ZSM-48, Pt/ZSM-12 and Pt/SAPO-11. Further details and examples of suitable molecular sieves and dewaxing conditions are for example described in WO-A-9718278, U.S. Pat. No. 5,053,373, U.S. Pat. No. 5,252,527 and U.S. Pat. No. 4,574,043.

Catalytic dewaxing conditions are known in the art and typically involve operating temperatures in the range of from 200 to 500° C., suitably from 250 to 400° C., hydrogen pressures in the range of from 10 to 200 bar, preferably from 15 to 100 bar, weight hourly space velocities (WHSV) in the range of from 0.1 to 10 kg of oil per litre of catalyst per hour (kg/l/hr), suitably from 0.2 to 5 kg/l/hr, more suitably from 0.5 to 3 kg/l/hr and hydrogen to oil ratios in the range of from 100 to 2,000 normal litres of hydrogen per litre of oil. By varying the temperature between 280 and 380° C. at a pressure of between 15-100 bars, in the catalytic dewaxing step it has been found possible to prepare base oils having different pour points varying from suitably lower than below the lowest measurable pour point, which is around −60° C. to up to 0° C.

After performing the pour point reducing treatment and if required lower boiling compounds formed during said treatment are suitably removed, preferably by means of a vacuum distillation, flashing step or a stripping step or combinations of said steps. One or more base oils grades may be obtained by distillation of the dewaxed product. Preferably such a distillation is performed in one distillation step performed under low pressure.

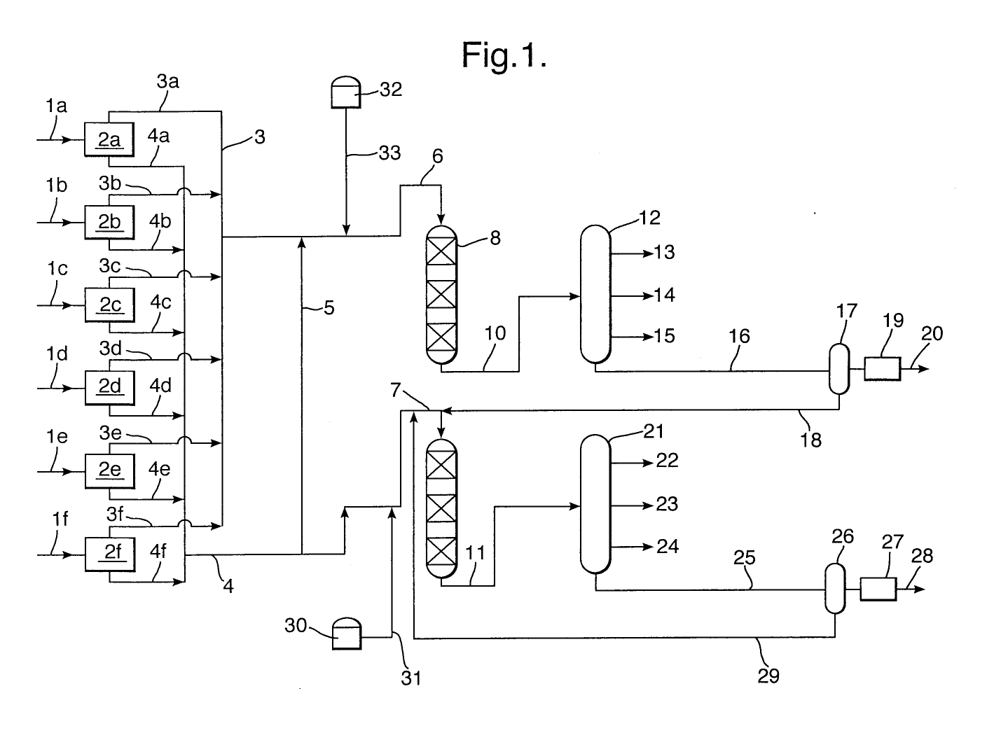

FIG. 1 shows a process scheme in which the process according to the present invention may suitably be carried out. In FIG. 1 a mixture of carbon monoxide and hydrogen (1a-1f) is fed to 6 parallel-operated Fischer-Tropsch synthesis reactors (2a-2f). The Fischer-Tropsch products as prepared in said reactors are typically recovered as a liquid product (4a-4f) and as gaseous products (3a-3f). The gaseous products (3a-3f) are condensed and combined to form stream (3), which is preferentially used to form composition (i) (6). The liquid products are combined to stream (4) which in part is combined with stream (3) and in part used to form composition (ii) (7). Composition (i) is converted in hydroprocessing reactor (8) to yield an effluent (10). Composition (ii) is converted in hydroprocessing reactor (9) to yield an effluent (11). The reactors (8, 9) are provided with stacked beds of catalyst as schematically drawn. The effluents (10, 11) of the reactors (8, 9) are separately distilled in distillation columns (12, 21) operating at atmospheric conditions. In these columns different distillate products are obtained, namely light overhead products (not shown), a naphtha product (13, 22), a kerosene product (14, 23), a gas oil product (15, 24) and a distillation residue fraction (16, 25). These two residue fractions can be finished iso-paraffinic base oil products having the required pour point. Optionally the heavy ends can be separated from these products in vacuum distillation columns (17, 26), which may also be a combined distillation. The distillation residues (18, 29) comprising of fractions boiling above the main grade base oil products, suitably boiling above 500° C., are recycled to preferentially reactor (9). They will thus form part of composition (ii) (7). FIG. 1 also shows an optional catalytic dewaxing units (19, 27), which may also be one reactor, to further decrease the pour point of the base oil products (20, 28). FIG. 1 also shows a cold slops tank (32) and a hot slops tank (30) which contain additional feed (33) or (31) for respectively composition (i)(6) and (ii)(7).

The invention will be illustrated by the following non-limiting examples.

The invention will be illustrated by the below Examples.

### EXAMPLE 1

Two feed compositions were obtained from a Fischer-Tropsch feed having the properties as listed in Table 1. Compositions (i) and (ii) were each subjected to a separate hydroconversion/hydroisomerisation step wherein the feed was contacted with a 0.8 wt % platinum on amorphous silica-alumina carrier. The conditions in the hydrocracking step were: a feed Weight Hourly Space Velocity (WHSV) of 1.0 kg/l.h, no recycle, and hydrogen gas rate=1000 Nl/kg feed, total pressure=32 bar. The reactor temperature was adjusted to achieve a substantial same conversion. The hydrocracker effluents were analysed and the yields and properties for the middle distillate and waxy Raffinate products are listed in Table 2.

TABLE 1

Sample

| | |
|---|---|
| Composition (i) | Composition (ii) |

(% weight fraction boiling below
listed boiling point)
(% weight)

| | | |
|---|---|---|
| 370° C. | 17.9 | 18.1 |
| (~C22) | | |
| 540° C. | 46.3 | 38.2 |
| (~C43) | | |

Weight ratio of compounds boiling
above 540° C. and compounds
boiling between 370° C. and 540° C.

| | | |
|---|---|---|
| 540° C.+/ | 1.9 | 3.1 |
| (370° C.-540° C.) | | |

TABLE 2

Reactor

| | 1 | 2 |
|---|---|---|
| | Composition | Composition |
| Feed | (i) | (ii) |
| Reactor Temperature, | 333 | 336 |
| ° C. | | |
| Fraction boiling below | 57.3 | 57.2 |
| 370° C. (wt %) | | |
| Fraction boiling | 12.2 | 13.4 |
| between C5 and 150° C. | | |
| (wt %) | | |
| Fraction boiling | 8.5 | 8.3 |
| between 150 and 200° C. | | |
| (wt %) | | |
| Fraction boiling | 35.1 | 33.4 |
| between 200 and 370° C. | | |
| Fraction boiling | 20.4 | 21.8 |
| between 370 and 540° C. | | |
| (wt %) | | |
| Pour point of fraction | +30° C. | +27° C. |
| boiling between 370 | | |
| and 540° C. (° C.) | | |
| Cloud point of | +43° C. | +38° C. |
| fraction boiling | | |
| between 370 and 540° C. | | |
| (° C.) | | |

As can be seen by comparing the results in Table 2 is that the Waxy Raffinate yield on Fischer-Tropsch derived product (feed) is significantly higher (7% relative increase) in Reactor 2 (=21.8 wt %) as compared to Reactor 1 which processes the lighter composition (i) (=20.4 wt %). Both Pour and Cloud Points of the Waxy Raffinate fraction are significantly better for the Waxy Raffinate derived in Reactor 2 (PP=+27° C. and CP=+38° C.), as compared to the Waxy Raffinate derived in Reactor 1 (PP =+30° C. and CP =+43° C.). In Reactor 1 however the yield to gas oil is significantly higher than in Reactor 2.

Further optimizing can be achieved by increasing the conversion in Reactor 1 to increase the yield and its iso-paraffin content to gas oil boiling between 200 and 370° C. and decrease the conversion in Reactor 2 to optimise the 370-540° C. yield while maintaining the pour point of this fraction at a value below 45° C. making it suitable for further pour point reduction processing to prepare base oils.

1. A process to prepare an iso-paraffinic product having a carbon range of Cx to Cy and a iso-paraffinic product having a carbon range of Cn to Cm from a Fischer-Tropsch derived feed comprising the following steps,
(a) obtaining from the Fischer-Tropsch derived feed at least two different compositions (i) and (ii), which composition (i) has a greater fraction of compounds in the carbon range of C2n to C2m than composition (ii) and composition (ii) has a greater content of C2x to C2y than composition (i), each fraction containing at least 5 wt % based on the whole fraction of material boiling above 370° C.;
(b) performing separately a hydroconversion/hydroisomerisation step on feed compositions (i) and (ii) and isolating from the thus obtained effluents the iso-paraffinic product having a carbon range of Cx to Cy and the iso-paraffinic product having a carbon range of Cn to Cm.

2. The process according to claim 1, wherein the Fischer-Tropsch derived feed has at least 50 wt % of compounds having at least 30 carbon atoms.

3. The process according to claim 1, wherein the weight ratio of compounds having at least 60 or more carbon atoms and compounds having at least 30 carbon atoms of the Fischer-Tropsch derived feed is at least 0.4.

4. The process according to claim 1, wherein n is between 14 and 16, m is between 20 and 25, x is between 20 and 25 and y is between 40 and 50.

5. The process according to claim 1, wherein n is between 10 and 12, m is between 14 and 16, x is between 14 and 16 and y is between 20 and 25.

6. The process according to claim 1, wherein n is 5, m is between 9 and 12, x is between 14 and 16 and y is between 20 and 25.

7. The process according to claim 1, wherein n is between 9 and 12 m is between 16 and 20, x is between 16 and 20 and y is between 20 and 25.

8. The process according to claim 1, wherein the weight ratio of C2n to C2m over Cn to Cm is greater than 1.5 in composition (i).

9. The process according to claim 1, wherein the weight ratio of C2x to C2y over Cx to Cy is greater than 1.5 in composition (ii).

10. The process according to claim 1, wherein the Fischer-Tropsch derived feed is obtained in two or more parallel operated Fischer-Tropsch synthesis reactors as a liquid and a gaseous product, wherein the gaseous product is condensed to form a condensed product and wherein step (a) is performed by adding the condensed products in a greater amount to composition (i) than to composition (ii).

11. The process according to claim 1, wherein the Fischer-Tropsch derived feed is obtained in two or more parallel operated Fischer-Tropsch synthesis reactors thereby obtaining at least two different Fischer-Tropsch products wherein the products comprises a C₂₀⁺ fraction having different ASF-alpha values (Anderson-Schulz-Flory chain growth factor), and wherein step (a) is performed by providing composition (i) with more of the Fischer-Tropsch product having the lower ASF-alpha value and composition (ii) with more of the Fischer-Tropsch product having the higher ASF-alpha value.

12. The process according to claim 11, wherein the lower ASF value is below 0.94 and the higher ASF value is above 0.94.

13. The process according to claim 1, wherein step (a) is performed by splitting the Fischer-Tropsch product into three parts (aa,bb,cc), wherein one part (aa) is separated into a high boiling part and a lower boiling part by means of distillation or flashing and wherein the lower boiling part is added to part (bb) and the high boiling part is added to (cc) to obtain compositions (i) and (ii) respectively.

14. The process according to claim 1, wherein step (a) is performed by adding cold slops to a part of the Fischer-Tropsch derived feed to obtain composition (i).

15. The process according to claim 1, wherein step (a) is performed by selectively adding part or all of the unconverted fraction obtained in step (b) to the Fischer-Tropsch feed to obtain composition (ii).

16. The process according to claim 1, wherein step (b) is performed in two parallel and continuously operated reactors for respectively feed composition (i) and (ii).

17. The process according to claim 1, wherein the conversion is higher when performing step (b) for composition (i) than when performing step (b) for composition (ii).

18. The process according to claim 1, wherein step (a) is performed by adding hot slops to a part of the Fischer-Tropsh derived feed to obtain composition (ii).
